(19) United States
(12) Patent Application Publication
(10) Pub. No.: US 2012/0201903 A1
(43) Pub. Date: Aug. 9, 2012

(54) CYTOPROTECTIVE OR THERAPEUTIC PLANT COMPOSITION

(75) Inventor: Donald B. Bowman, Waldport, OR (US)

(73) Assignee: Oregon Health & Science University, Portland, OR (US)

(21) Appl. No.: 13/501,075

(22) PCT Filed: Oct. 12, 2010
(86) PCT No.: PCT/US10/52375

§ 371 Date: Apr. 9, 2012

Related U.S. Application Data
(60) Provisional application No. 61/250,165, filed on Oct. 9, 2009.

(51) Int. Cl. A61K 33/14; A61K 36/31; A61P 17/02; A61P 37/08; A61P 29/00; A61K 36/8962; A61P 35/00

(52) U.S. Cl. 424/680; 424/754; 424/755

(57) ABSTRACT

Embodiments herein provide plant compositions, and, more specifically, a plant composition including Allium vineale or an extract thereof. Other compositions are also provided, as well as methods of formulating the compositions, and methods of using the compositions to treat or prevent conditions such as cancer, allergies, and inflammation, or to promote cytoprotection or accelerate wound healing.

### CROSS REFERENCE TO RELATED APPLICATIONS

The present application claims priority to U.S. Provisional Patent Application No. 61/250,165, filed Oct. 9, 2009, entitled “Cytoprotective or Therapeutic Plant Composition Including Allium Vineale,” the entire disclosure of which is hereby incorporated by reference in its entirety.

### TECHNICAL FIELD

Embodiments herein relate to the field of plant compositions, and, more specifically, to a cytoprotective or therapeutic plant composition, such as one including Allium vineale, methods of formulating such compositions, and applications of such compositions.

### BACKGROUND

Allium vineale is a perennial bulbflower in the genus Allium that is native to Europe, northern Africa and western Asia. A. vineale has been introduced in Australia and North America, where it has become an invasive species. It produces a garlic odor and taste, and it is difficult to control once established.

A. vineale contains phytochemicals that have therapeutic effects; however, it is difficult to transport these compounds into cells in sufficiently high concentrations to be therapeutic.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates an HPLC chromatogram (350 nm) of Allium vineale leaf extract in accordance with various embodiments.

FIG. 2A illustrates an HPLC chromatogram (280 nm) of A. vineale leaf extract treated with 1 N HCl. FIGS. 2B-2E show UV data of the first four major peaks of FIG. 2A, in accordance with various embodiments.

FIG. 3 illustrates an HPLC chromatogram of Sephadex LH-20 fraction 31, which contains a quercetin-O-glycoside, in accordance with various embodiments.

FIG. 4 illustrates comparative growth of K562 cells with composition 462 over a 120 hour time period in accordance with various embodiments.

FIG. 5 illustrates comparative growth of K562 cells with composition 462 at 120 hours in accordance with various embodiments.

FIG. 6 illustrates comparative growth of K562 cells and dose response of composition 462 at 72 hours in accordance with various embodiments.

FIG. 7 shows results from a mouse study using PC3 xenografts illustrating individual components and the full 462 composition effect on tumor growth; OS is olive oil/salt, EOS is Vitamin E/olive oil/salt, GOS is A. vineale/olive oil/salt, and Garl 462 is the full composition.

FIG. 8 represents tumor weight from PC3 xenografts; net tumor weights were collected at necropsy, 462-0-2-4 represents the weight of the tumors in 3 of the 5 mice that displayed complete growth inhibition and partial resorption.

FIG. 9 illustrates comparative growth of K562 cells with quercetin at 72 hours in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide a cytoprotective plant composition including Allium vineale. Some embodiments provide for treatment of various conditions with A. vineale or with one or more components extracted from A. vineale, alone or in combination with one or more additional components. An example embodiment provides a pharmacologically active emulsion that demonstrates transdermal delivery, and that may be used for the treatment and prevention of various conditions, such as cancer, allergies, and inflammatory diseases, or to promote cytoprotection or accelerate wound healing.

In an embodiment, an extraction of A. vineale may be obtained using a two-phase aqueous and organic water-immiscible solvent system composed of heated (boiling) salt water, olive oil or omega-3 fish oil, vitamin E, and polysorbate 80, a pharmaceutically acceptable surfactant. The salt water, oil, vitamin E, surfactant, and extracted plant compounds spontaneously form a solubilized emulsion.

In one specific, non-limiting example, an A. vineale extract emulsion may be generated according to the following procedure. About 30-70 g A. vineale leaves (for example, about 50 g) are added to 1000 ml boiling distilled water with about 9-30 g NaCl (for example, about 10 g NaCl. Preferably, the A. vineale leaves are fresh fall or spring green growth, however other plant tissue, e.g., bulbs, aerial bulblets, sprouts, etc., and dried plant material also may be used. While continuing to boil the mixture, about 4-8 gelcaps, 400 IU each (Nature Made Nutritional Products, Mission Hills, Calif.) of water solubilized synthetic vitamin E (dl alpha-tocopheryl acetate) that contain polysorbate 80 are added, followed by about 15-30 ml olive oil (pure or extra virgin). The mixture is then boiled for about 45 minutes, which reduces the volume down to about 300-500 ml. The ingredients may be added simultaneously or in series. For instance, in some examples, the olive oil is not added to the mixture until the final 10-15 minutes of boiling. The exemplary protocol described above produces an emulsion referred to as composition 462 or NAT 462.

The mixture is then filtered, removing plant material and excess oil. The product (filtrate) has a slightly hazy light yellow color and transmits a narrow beam of light (positive Tyndall effect) and does not spin down by ultracentrifuge. Without being bound by theory, it is believed that the NaCl contributes to solubilization of the emulsion because without added salt, a milk-like consistency results that is hard to filter and that does not penetrate the skin as effectively.

The water solubilized vitamin E capsules used in this exemplary protocol contain dl-alpha tocopheryl acetate, polysorbate 80, water, non-bovine gelatin, and glycerin. Methods of making water solubilized vitamin E are known, and may be found in U.S. Pat. No. 6,180,130, which is incorporated by reference herein. Briefly, vitamin E acetate and polysorbate 80 are mixed in a 2:3 ratio and heated to about 40° C. to make a homogeneous mixture. This mixture is then diluted 1:1 with water at about 30-35° C. to form a gel-like consistency.

In some embodiments, other salt sources or saline solutions may be substituted for the salt water used in the exemplary protocol, for instance lactated Ringer's solution, acetated Ringer's solution, phosphate buffered saline (PBS), TRIS-buffered saline (TBS), Hank's balanced salt solution (HBSS), Earle's balanced salt solution (EBSS), standard saline citrate (SSC), HEPES-buffered saline (HBS), and Gey's balanced salt solution (GBSS).

In other embodiments, α-, δ-, γ-, or δ-tocopherol, or corresponding tocotrienols, or a mixture thereof, may be substituted for or used in combination with the vitamin E acetate/succinate of the exemplary protocol.

In still other embodiments, other surfactants may be substituted for polysorbate-80. Specific, non-limiting examples of surfactants include perfluorooctanoate (PFOA or PFO), perfluorooctanesulfonate (PFOS), sodium dodecyl sulfate (SDS), ammonium lauryl sulfate, sodium laureth sulfate, alkyl benzene sulfonate, cetyl trimethylammonium bromide (CTAB), cetylpyridinium chloride (CPC), polyethoxylated tallow amine (POEA), benzalkonium chloride (BAC), benzethonium chloride (BZT), dodecyl betaine, cocamidopropyl betaine, coco ampho glycinate, alkyl poly(ethylene oxide), alkylphenol poly(ethylene oxide), octyl glucoside, decyl maltoside, cetyl alcohol, oleyl alcohol, cocamide MEA, cocamide DEA, Tween 20, and dodecyl dimethylamine oxide.

While olive oil and omega-3 fish oil are mentioned as example oils, other suitable oils may be used, as desired, such as other vegetable based oils, etc.

In an example embodiment, a composition may comprise 10-75% by weight A. vineale in whole form or as an extract; 0-10% by weight sodium chloride (NaCl); 5-35% by weight Vitamin E; 0-20% by weight surfactant; and 0-10% by weight oil. In certain forms, such as an emulsion, water may be present in the final composition. In addition, other components may be included as discussed further below.

An embodiment provides a composition comprising a therapeutically effective amount of Allium vineale or an extract thereof; and a therapeutically effective amount of an α-, β-, γ-, or δ-tocopherol or tocotrienol, or a mixture thereof. In accordance with another embodiment, there is provided a composition comprising a therapeutically effective amount of Allium vineale or an extract thereof; a therapeutically effective amount of sodium chloride; a therapeutically effective amount of vitamin E; a therapeutically effective amount of polysorbate 80; and a therapeutically effective amount of olive oil or omega-3 fish oil.

In an embodiment, one or more compounds of A. vineale may be extracted, such compounds selected from allyl sulfides, allyl cysteines, 5-hydroxymethyl-2-furaldehyde, hydroxyacetyl furan, quercetin, quercetin-O-rhamnoside, quercetin-O-glycoside, kaempferol, and kaempferol-O-glucoside.

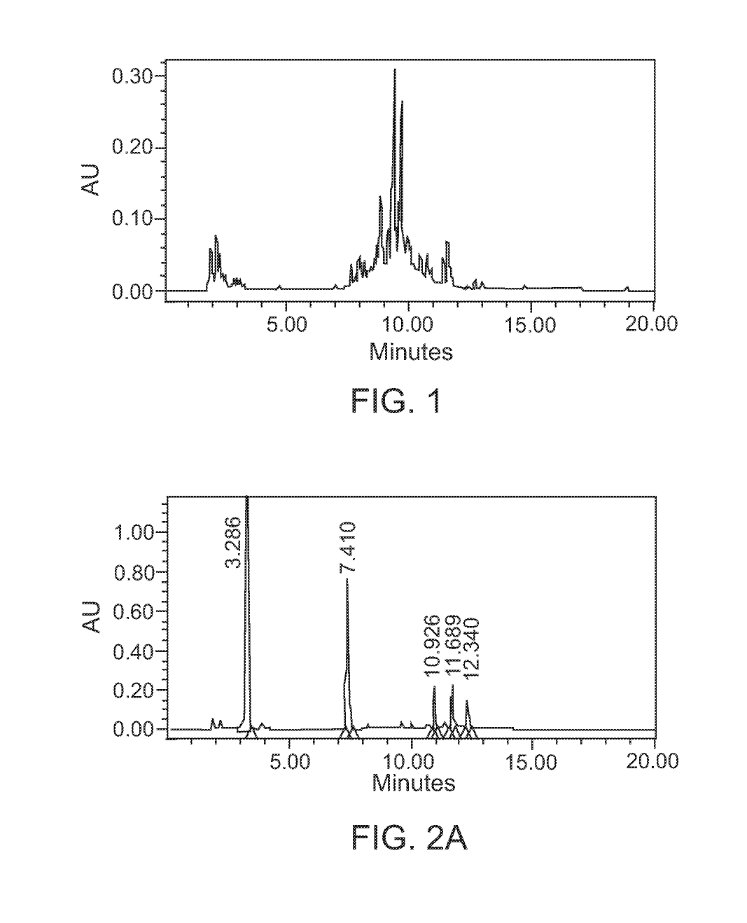

In one embodiment, the chemical composition of the A. vineale extract was analyzed by high-performance liquid chromatography (HPLC) as follows. Briefly, A. vineale leaves were lyophilized for one week and the dried materials were ground. Ground leaves (1.0 g) were extracted with a mixture of methanol and water (2:1 v/v, 30 mL) and the crude extract was analyzed by HPLC with UV detection (FIG. 1).

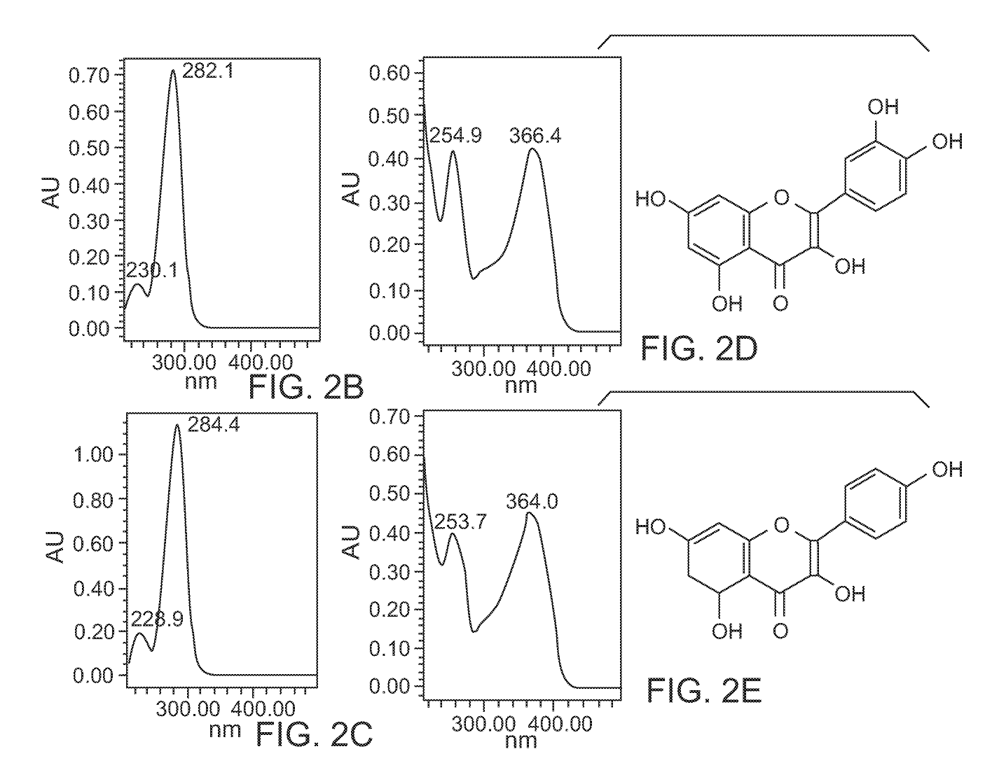

A portion of the extract was then treated with 1 N HCl to hydrolyze glycosides. When analyzed using a combination of Sephadex LH-20 column chromatography, semi-preparative HPLC, two-dimensional nuclear magnetic resonance (NMR), and high-resolution mass spectrometry, the first four major peaks of the hydrolyzed sample corresponded to 5-hydroxymethyl-2-furaldehyde, hydroxyacetyl furan, quercetin, and kaempferol (FIG. 2A). The peak at 12.3 minutes in FIG. 2A represents a side product of glycoside hydrolysis.

FIGS. 2B-2E show UV data of the first four major peaks of FIG. 2A. The compounds 5-hydroxymethyl-2-furaldehyde (FIG. 2B) and hydroxyacetyl furan (FIG. 2C) produced a UV maximum at 282 nm and 284 nm respectively. Quercetin (FIG. 2D) and kaempferol (FIG. 2E) were identified by comparison with authentic markers.

Without being bound by theory, it is believed that 5-hydroxymethyl-2-furaldehyde and hydroxyacetyl furan are thermal degradation products of plant sugars removed by hydrolysis from the extracted polyphenolic compounds. These thermal degradation products are thought to be produced by the heat used in boiling the extraction mixture. It also is believed that the highly reactive aldehyde and C═O groups of these compounds bind to SH groups of proteins and enzymes involved in cell division.

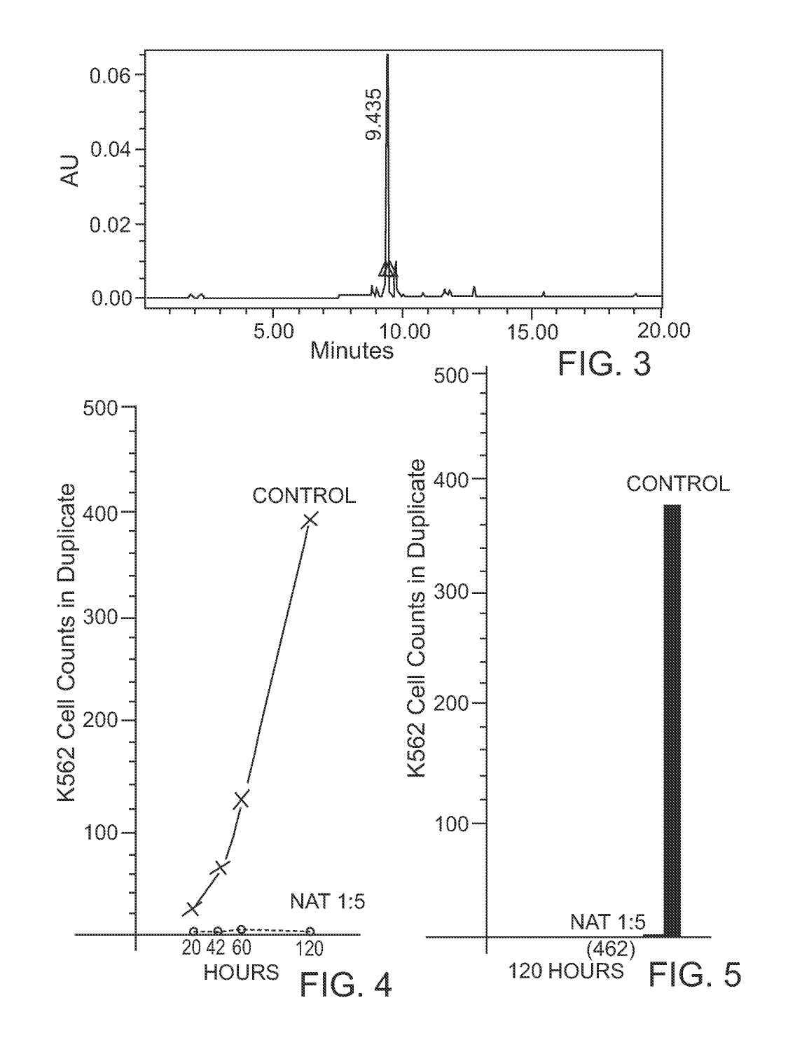

The remainder of the leaf extract was fractionated by column chromatography using Sephadex LH-20 as the stationary phase and methanol as the mobile phase. Forty fractions (11 mL each) were collected and analyzed by HPLC-UV. Fraction 31 (FIG. 3) contained the major peak component of FIG. 1. This peak fraction was treated with 1 N HCl and yielded quercetin, indicating that the major peak component of the leaf extract is quercetin-O-glycoside. Other components of composition 462 include kaempferol-O-glucoside and quercetin-O-rhamnoside, both of which exhibit HPLC peaks with MW around 488.

Quercetin and kaempferol are both polyphenolic (flavonol) compounds that appear to have anti-inflammatory activity through down-regulation of the NF-KB pathway. In addition, without being bound by theory, quercetin is believed to inhibit the activity of tyrosine kinases and polyphenolics such as quercetin and kaempferol, are reported to activate Nrf2 signaling. Containing sulforaphane (SFN), 4625 and 462FS also act through the inhibition of histone deacetylase (HDAC) and also induce Nrf2-phase 2 enzymes.

The 462 composition also appears to induce autophagy. Composition 462 appears to induce cancer cell death by selectively regulating oxidative stress in cancer cells through inhibition of the Nrf2 pathways and phase I and II antioxidant enzyme SOD1 and GSTP1. The increase in oxidative stress in cancer cells was accompanied with an upward shift in the beclin1/Bcl2 ratio forcing the cells to undergo cell death and activation of autophagy. In particular, treated PC3 cells demonstrated a 2.3 fold increase in the Beclin1/Bcl2 relationship and treated PC3 prostate cancer xenografts in mice demonstrated combined autophagy, necrosis and apoptosis. By enhancing clearance of neurotoxic mutant proteins, inducing autophagy also may hold promise for treating Huntington's disease and other presently untreatable neurodegenerative diseases. Autophagy may also play a role in anti-microbial host defense. Without being bound by theory, it is believed that composition 462 targets combined AKT/mTOR and ERK MAPK signaling to inhibit hormone-refractory prostate cancer. Further activities may include the inhibition of DBA-topoisomerase by the flavonoids (e.g., kaempferol glycosides) and the inhibition of mTOR.

As described above, during testing, the A. vineale emulsion (e.g., composition 462) was shown to halt the growth of, and trigger apoptosis of, tumor cells. In one embodiment, K562 cells (chronic myelogenous leukemia, human, American Type Culture Collection, Rockville, Md.: ATCC #CCL-243) were cultured in RPMI medium 1640 with L-glutamine (Gibco BRL, Grand Island, N.Y.), with streptomycin sulfate (100 mg/L, Sigma, St. Louis, Mo.), penicillin G sodium salt (100,000 U/L, Sigma), and 10% Fetal Bovine Serum (Hyclone Laboratories, Logan, Utah) in 25 cm² vented tissue culture flasks (Corning, Corning, N.Y.), and incubated at 37° C. in 5% CO₂ (Fisher Scientific). Cell manipulations were performed using a Nuaire Biological Safety Cabinet (Plymouth, Minn.), individually wrapped sterile disposable serological pipettes (Fisher Scientific, Pittsburgh, Pa.), and a Pipet-Aid (Drummond, Broomall, Pa.). Cell viability was assessed by trypan blue exclusion and cell numbers were determined with a hemocytometer using a compound microscope.

Cells were inoculated uniformly at a target concentration of approximately 100,000 cells/ml in 5 ml volumes in 25 cm² flasks; each sample inoculated in duplicate. For the test flasks, the A. vineale emulsion (e.g., composition 462) was added at a 1/5 dilution: e.g., 1 ml of medium was withheld from each test flask and replaced with 1 ml of composition 462. Viable cell counts were expressed as the actual number counted in the hemocytometer. The number of cells/ml is this number multiplied by 25×10³.

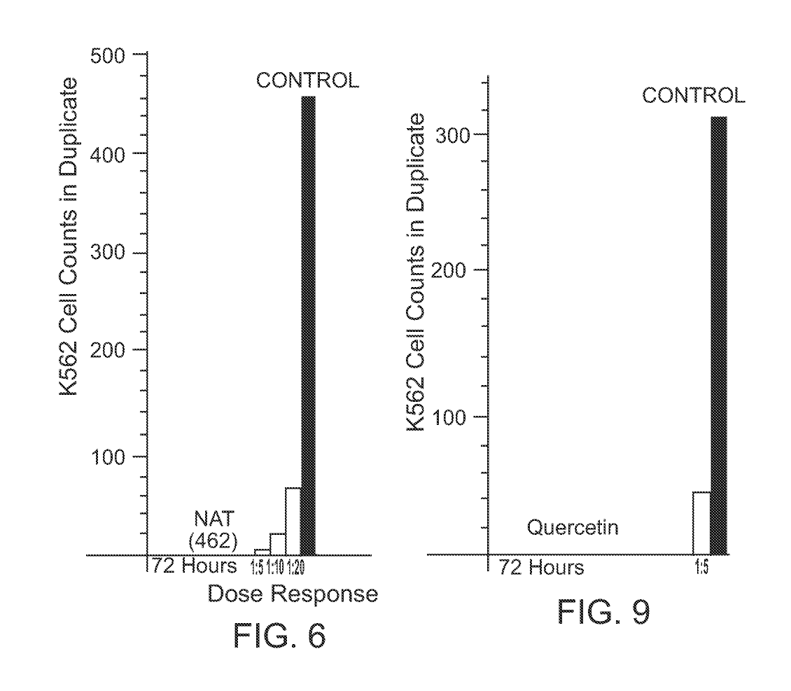

As shown in FIG. 4, the growth of K562 cells was reduced dramatically by the 462 composition over the course of a 120-hour time period as compared to untreated cells. Similarly, as shown in FIG. 5, composition 462 strongly inhibited the number of K562 cells at the 120-hour time point as compared to untreated cells. FIG. 6 illustrates the comparative growth of K562 cells and dose response of the 462 composition, and demonstrates that even a 1:20 dilution of composition 462 inhibited cell growth.

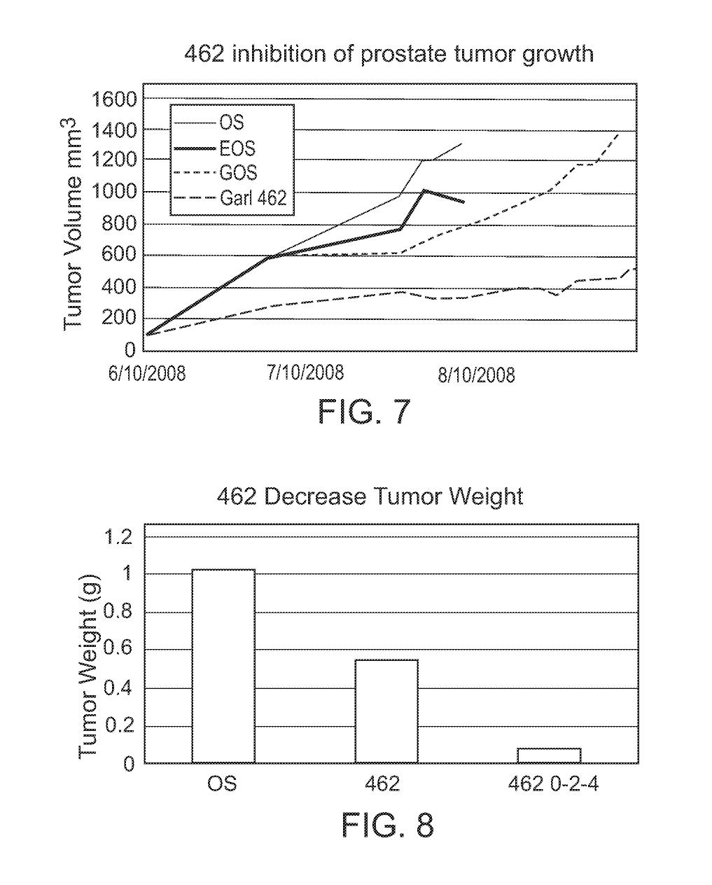

FIG. 7 shows results from a mouse study using PC3 xenografts illustrating individual components and the full 462 composition effect on tumor growth; OS is olive oil/salt, EOS is Vitamin E/olive oil/salt, GOS is A. vineale/olive oil/salt, and Garl 462 is the full composition.

Right and left flanks of 6-week old male Nu/Nu mice (Charles River, Wilmington, Mass.) were inoculated with 1.2×10⁶ PC-3 cells in 0.3 mL RPMI-1640 medium containing 50% Matrigel (BD Biosciences, Bedford, Mass.). Once tumors began to show, mice were divided into 4 groups of five mice: OS, EOS, GOS, and composition 462 treated. Each mouse was injected subcutaneously with 600 μl of its treatment composition five days a week. Two days a week, body weights and tumor measurements were taken. Tumor sizes were determined by measuring length, width and thickness and applying the volumetric equation [V=W×L×T×π/6]. At the conclusion of the experiment, xenograft tumors were excised and ⅓ was fixed for histology for H&E staining and TUNEL, the rest of the tumor was processed for RNA (in Tri-Reagent) and protein. RNA and protein samples were snap frozen in liquid nitrogen and stored at −80° C.

Treatment with composition 462 significantly decreased the growth of the tumor xenografts over time when compared to OS treated mice (FIG. 7). Furthermore, a subset of 3 out of the total 5 mice in the group treated with composition 462 showed exceptional inhibition of growth compared to the entire group treated with composition 462 and to the control. At sacrifice, the mean tumor volume for the complete 462-treated group was 520.7 mm³+/−37 compared to 1516.5 mm³+/−311.6 for the control. When examining only the 462-treated subset, the mean tumor volume was even further decreased with a value of 150.6 mm³+/−11.03 (FIG. 7). Composition 462 had a dramatic effect on tumor weights with the complete 462 group having a mean tumor weight of 0.55 g+/−0.01 compared to 1.17 g+/−0.22 for the control. Again, the subset of 462-treated tumors showed a further decrease of tumor weight with a mean tumor weight of 0.06 g+/−0.01. Both the 462-treated group and the control group showed no dramatic differences in body mass thus eliminating the possibility of gross toxicity with treatments. Analysis of tumor weights at necropsy further confirmed the dramatic decrease in tumor volume in mice treated by composition 462 (FIG. 8).

FIG. 8 represents tumor weight from PC3 xenografts; net tumor weights were collected at necropsy, 462-0-2-4 represents the weight of the tumors in 3 of the 5 mice that displayed complete growth inhibition and partial resorption.

FIG. 9 demonstrates the effect of quercetin, an active ingredient found in A. vineale, alone on the growth of K562 cells.

Thus, in composition 462, the A. vineale-derived active ingredients appear to act synergistically with vitamin E.

In the above-discussed preparation, it also is possible that small amounts of organosulfur compounds may be extracted from A. vineale, but it is believed that Alliinase, a plant enzyme that converts precursor organosulfur compounds to active forms, may be inhibited by the boiling in saline, limiting release of these more familiar Allium plant compounds.

A. vineale is described throughout; however, in embodiments other Allium species may be substituted.

In some embodiments, the A. vineale composition is referred to as 462FS, which includes A. vineale flavonoids and sulforaphane.

In still other embodiments, the composition is referred to as 462S, in which broccoli sprouts, which are high in sulforaphane, are substituted for the A. vineale leaves during formation of the composition/emulsion.

Thus, in an embodiment, there is provided a composition comprising a therapeutically effective amount of broccoli sprouts or an extract thereof; a therapeutically effective amount of vitamin E; and a surfactant. In embodiments, a surfactant may or may not be present. If present, a suitable surfactant is polysorbate 80 and others described elsewhere herein.

In an embodiment, the A. vineale compositions described herein may be used to treat or prevent a variety of medical conditions, for instance, cancer, allergies, and inflammatory diseases. Such compositions may also be used to accelerate wound healing. For purposes of describing embodiments herein, the term “treat” or “treatment” refers to any therapy that reduces or ameliorates a symptom of a disease or disorder, such as a reduction in the number, growth, or growth rate of a quantity of cancer cells. As used herein, the term “treatment” may refer to a therapy that causes a cure or reduction of a symptom to a normal state or condition, or it may refer to a therapy that causes only a partial reduction in a symptom, such as a 10%, 20%, 30%, 40%, 50%, 60%, 70%, 80%, or 90% reduction.

Thus, an embodiment herein provides a method of treating a cancer, allergy, or inflammatory disease in a subject comprising selecting a subject having at least one of a cancer, allergy, or inflammatory disease; and administering to the subject a therapeutically effective amount of a composition as described herein, thereby treating the cancer, allergy, or inflammatory disease in the subject. There is also a method provided for accelerating wound healing in a subject comprising selecting a subject having at least one tissue wound; and administering to the subject a therapeutically effective amount of a composition as described herein, applied topically, thereby accelerating healing of the tissue wound.

For instance, in an embodiment, an A. vineale composition may be administered to a subject locally or systemically. In certain embodiments, the composition is administered in the form of a self-emulsifying drug delivery system (SEDDS). A SEDDS is a drug delivery system that uses an emulsion achieved by chemical rather than mechanical means. That is, the emulsion is formed by an intrinsic property of the drug formulation, rather than by special mixing and handling. Emulsions have significant uses in drug delivery, and SEDDS are the most effective of these systems. SEDDS are of particular value in increasing the absorption of lipophilic drugs administered transdermally or by mouth.

SEDDS offer numerous advantages: spontaneous formation, ease of manufacture, thermodynamic stability, and improved solubilization of bioactive materials. Improved solubility contributes to faster release rates and greater bioavailability. Greater bioavailability means that less drug may be used, which results in lower cost and reduces side effects and toxicity.

For topical use, the SEDDS described herein typically are formulated as liquids, creams, or ointments. For parenteral use, the SEDDS typically are provided as liquids. For oral use, SEDDS may be formulated as liquids or solids; the solids being packaged in capsules or tablets. In terms of bioavailability, liquid SEDDS are superior to solid SEDDS, which are superior to conventional tablets.

One of the advantages of the A. vineale SEDDS compositions described herein is that they have the ability to move combinations of active phytochemicals into cells in sufficiently high concentrations to be therapeutic. These therapeutically active preparations represent one-pot hydrolysis-extractions of flavonol glycosides converted to active aglycone forms accompanied by the conversion of hydrolyzed-off sugars to additional ingredients, all incorporated in SEDDS. Additionally, the combinational effects of the A. vineale compounds and vitamin E appear complementary and synergistic; these specific combinations are effective in treating diseases, even when the individual compounds are ineffective or less effective for that purpose. The extraction process provides both an effective combination of components that act in concert and a drug delivery vehicle.

In embodiments, the A. vineale compositions described herein may be used to treat or prevent a variety of cancers, for instance, colorectal cancer, breast cancer, ovarian cancer, prostate cancer, leukemia, malignant glioma, squamous cell carcinoma, basal cell carcinoma, actinic keratosis, and other skin tumors; allergy, including seasonal allergy, intractable hay fever, and allergic reactions with associated pain, inflammation, and itching; inflammation and inflammatory diseases, including joint inflammation and swelling, tenosynovitis, arthritis, and itching/psoriasis. The compositions also may be used for neuroprotection, including the treatment and prevention of Huntington's disease, and they also may accelerate healing, for instance in corneal abrasions, unsutured lacerations, oral canker sores, and thermal burns. Other inflammatory diseases and disorders that may be treated or prevented using the A. vineale compositions described herein include asthma, chronic obstructive pulmonary disease (COPD), septic shock, non-healing ulcers, surgical wounds, cerebral edema, stroke, myocardial infarction, traumatic head injury, reperfusion injury, restenosis, and tissue rejection.

Compositions described herein may be used to treat benign skin lesions, e.g., seborrheic keratosis and verruca vulgaris (HPV common wart) may be reduced or eradicated using a disclosed composition, or solar lentigo (liver spots) may be reduced or caused to fade. Thus, compositions herein may be provided in a cosmetic form or to impart a cosmetic effect.

In other embodiments, the SEDDS described herein may function as drug carriers or cytoprotective adjuncts for standard cancer treatments.

In embodiments, the A. vineale compositions may be administered topically (for instance, to the skin or nasal mucosa), parenterally (for instance, subcutaneously, intravenously, or intramuscularly), or orally. The A. vineale compositions may be used alone or in combination with other anti-cancer or anti-inflammatory agents. In an embodiment, the A. vineale compositions may be administered in varying concentrations depending upon requirements of the patient, the severity, type, stage, grade, or location of the disease being treated, the route of administration, the particular A. vineale composition being used, and the general health of the subject.

Pharmaceutical compositions in accordance with embodiments of the disclosure may be prepared by combining the disclosed compounds with a solid or liquid pharmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier may be at least one substance that may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds disclosed herein dissolved in water and water-propylene glycol systems, optionally containing suitable conventional coloring agents, flavoring agents, stabilizers, and/or thickening agents.

In an embodiment, a pharmaceutical composition may be provided employing conventional techniques in unit dosage form containing effective or appropriate amounts of one or more active component. In embodiments, the quantity of active component (compound) in a pharmaceutical composition and unit dosage form thereof may be varied or adjusted widely depending upon the particular application, the potency of the particular composition, and the desired concentration. In an embodiment, the quantity of active component may range from about 0.5% to about 90% by weight of the composition.

In embodiments, in therapeutic use for treating, ameliorating, preventing, or combating a disease in a subject, the compound or pharmaceutical composition thereof may be administered topically, orally, or parenterally at a dosage to obtain and maintain a concentration or blood-level of active component in the subject undergoing treatment that is therapeutically effective. Various dosage ranges and administration schedules may be adopted for therapeutic treatment of animal or human subjects with the A. vineale compositions disclosed herein. In an embodiment, a therapeutically effective amount of dosage of active component may be in the range of about 0.1 to about 100 mg/kg, more preferably about 0.1 to about 10 mg/kg, of body weight/day. It is to be understood that the dosages may vary depending upon the requirements of the patient, the severity, stage, or grade of the disease, the route of administration, and the particular A. vineale composition being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose also may be divided into multiple doses for administration, for instance, two to four times per day.

As discussed above, embodiments provide methods for treating or preventing a variety of diseases and conditions. The methods include selecting a subject in need of treatment and administering to the subject a therapeutically effective amount of at least one composition disclosed herein.

As used herein, the term “therapeutically effective amount” includes a quantity of a specified compound (such as one of the A. vineale compositions disclosed herein, for instance the 462 composition) required to achieve a desired effect in a subject being treated. For instance, this may be the amount necessary to treat a cancer, such as a colorectal cancer, breast cancer, prostate cancer, leukemia, ovarian cancer, malignant glioma, squamous cell cancer, basal cell carcinoma, or other skin cancer in a subject, or a dose sufficient to prevent advancement, or to cause regression of a disease (such as cancer, as has been demonstrated in PC3 prostate cancer xenografts in SCID mice), or that is capable of relieving symptoms caused by a disease, such as pain, inflammation, neurological symptoms, or fatigue. In some embodiments, a therapeutically effective amount of an A. vineale composition is a dose that is sufficient to inhibit metastasis.

In other embodiments, a therapeutically effective amount of a composition is the amount necessary to treat an allergy or inflammatory disease, such as joint inflammation, tenosynovitis, intercostal neuritis, arthritis, psoriasis, or sigmoid diverticulitis in a subject, or a dose sufficient to prevent advancement, or to cause regression of a disease, or that is capable of relieving symptoms caused by a disease, such as pain or inflammation. In some embodiments, a therapeutically effective amount of an A. vineale composition is a dose that is sufficient to accelerate wound healing, such as in the case of corneal erosion, unsutured lacerations, oral canker sores, or thermal burns.

A compound or component in a composition may be defined as being present in a “therapeutically effective amount” even if that compound or component is not separately defined as an active component, but rather such a compound/component may impact one or more beneficial qualities of the composition, such as bioavailability.

In some embodiments, a composition herein may be administered in conjunction with one or more other compositions or treatments regimens.

In some embodiments, an A. vineale composition may be administered in conjunction with one or more other anti-cancer agents, such as such as alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and bbr3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, hydroxyurea, and mitomycin; topoisomerase inhibitors, such as topotecan and irinotecan; monoclonal antibodies, such as alemtuzumab, bevacizumab, cetuximab, gemtuzumab, rituximab, and trastuzumab; photosensitizers, such as aminolevulinic acid, methyl aminolevulinate, porfimer sodium, and verteporfin; cytokines, such as IL-2 and IL-27; and other agents, such as alitretinoin, altretamine, amsacrine, anagrelide, arsenic trioxide, asparaginase, bexarotene, bortezomib, celecoxib, denileukin diftitox, erlotinib, estramustine, gefitinib, hydroxycarbamide, imatinib, pentostatin, masoprocol, mitotane, pegaspargase, and tretinoin. In other embodiments, the composition is administered in conjunction with one or more anti-inflammatory agents, such as a steroidal or non-steroidal anti-inflammatory agent. In particular embodiments, an A. vineale composition may be used in its capacity as a drug delivery system or SEDDS for the effective delivery of one or more other drugs or compositions.

In some embodiments, an A. vineale composition may be administered systemically, whereas in other embodiments an A. vineale composition may be administered locally. An effective dose of a disclosed A. vineale composition may be administered systemically in a variety of ways. For instance, systemic administration may be by oral administration or by injection, for instance intravenous, intramuscular, or subcutaneous injection. Local (for instance topical) administration may include administration of a liquid, cream, or ointment to the skin or to the mucosa, for instance as a nasal formulation or ocular formulation.

An effective amount of an A. vineale composition may be administered in a single dose, or in multiple doses, for example daily, or every four, eight, or twelve hours, during a course of treatment. In one embodiment, a therapeutically effective amount of an A. vineale composition may be administered as a single pulse dose, as a bolus dose, or as pulse doses administered over time. In specific, non-limiting examples, pulse doses of an A. vineale composition may be administered during the course of a day, during the course of a week, during the course of a month, or over the course of years.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A composition comprising:
a therapeutically effective amount of Allium vineale or an extract thereof; and
a therapeutically effective amount of an α-, β-, γ-, or δ-tocopherol or tocotrienol, or a mixture thereof.

2. The composition of claim 1, wherein the therapeutically effective amount of Allium vineale or an extract thereof comprises a therapeutically effective amount of at least one of an allyl sulfide, an allyl cysteine, 5-hydroxymethyl-2-furaldehyde, hydroxyacetyl furan, quercetin, quercetin-O-rhamnoside, quercetin-O-glycoside, kaempferol, and kaempferol-O-glucoside.

3. The composition of claim 1, wherein the therapeutically effective amount of an α-, β-, γ-, or δ-tocopherol or tocotrienol, or a mixture thereof comprises a therapeutically effective amount of vitamin E.

4. The composition of claim 1, further comprising sodium chloride.

5. The composition of claim 1, further comprising a surfactant.

6. The composition of claim 5, wherein the surfactant is polysorbate 80.

7. The composition of claim 1, further comprising oil.

8. The composition of claim 7, wherein the oil is olive oil or omega-3 fish oil.

9. The composition of claim 1, further comprising a therapeutically effective amount of sulforaphane.

10. A composition comprising:
a therapeutically effective amount of Allium vineale or an extract thereof;
a therapeutically effective amount of sodium chloride;
a therapeutically effective amount of vitamin E;
a therapeutically effective amount of polysorbate 80; and
a therapeutically effective amount of olive oil or omega-3 fish oil.

11. The composition of claim 10, wherein the therapeutically effective amount of Allium vineale or an extract thereof comprises a therapeutically effective amount of at least one of quercetin, quercetin-O-rhamnoside, quercetin-O-glycoside, kaempferol, and kaempferol-O-glucoside.

12. The composition of claim 10, further comprising at least one of an allyl sulfide, an allyl cysteine, 5-hydroxymethyl-2-furaldehyde, and hydroxyacetyl furan.

13. The composition of claim 10, wherein the composition comprises a self-emulsifying drug delivery system (SEDDS).

14. The composition of claim 10, further comprising a therapeutically effective amount of sulforaphane.

15. A composition comprising:
a therapeutically effective amount of broccoli sprouts or an extract thereof;
a therapeutically effective amount of vitamin E; and
a surfactant.

16. The composition of claim 15, wherein the surfactant is polysorbate 80.

17. The composition of claim 15, further comprising at least one of a sodium chloride, olive oil, and omega-3 fish oil.

18. A method of making a composition, comprising combining Allium vineale leaves with salt water, an effective amount of olive oil or omega-3 fish oil, an effective amount of vitamin E, and an effective amount of a surfactant, thereby making the composition.

19. The method of claim 18, wherein combining the Allium vineale leaves with salt water, olive oil or omega-3 fish oil, vitamin E, and a surfactant, comprises boiling together the Allium vineale leaves, salt water, olive oil or omega-3 fish oil, vitamin E, and the surfactant to form an emulsion.

20. A method of treating a cancer, allergy, or inflammatory disease in a subject comprising:
selecting a subject having at least one of a cancer, allergy, or inflammatory disease; and
administering to the subject a therapeutically effective amount of the composition of claim 1, thereby treating the cancer, allergy, or inflammatory disease in the subject.

21. A method of accelerating wound healing in a subject comprising:
selecting a subject having at least one tissue wound; and
administering to the subject a therapeutically effective amount of the composition of claim 1, applied topically, thereby accelerating healing of the tissue wound.

22. A composition comprising:
a therapeutically effective amount of a species of Allium or an extract thereof; and
a therapeutically effective amount of an α-, β-, γ-, or δ-tocopherol or tocotrienol, or a mixture thereof.
